SEQUENCE LISTING (2 sequences)

SEQ ID NO: 1 - DNA, 20 bp, Artificial Sequence
  Synthetically Generated Sequence
ggggatggcaggcaactcat

SEQ ID NO: 2 - DNA, 20 bp, Artificial Sequence
  Synthetically Generated Sequence
atgagttgcctgccatcccc

(19) United States
(12) Patent Application Publication
(10) Pub. No.: US 2018/0074053 A1
(43) Pub. Date: Mar. 15, 2018

(54) TREATMENT OF DIABETES USING AGONISTS OF THE MAS-RELATED GPCR D

(71) Applicants: Zhongmin Ma, Tenafly, NJ (US); Bruce Edward Markham, Novi, MI (US)

(72) Inventors: Zhongmin Ma, Tenafly, NJ (US); Bruce Edward Markham, Novi, MI (US)

(21) Appl. No.: 15/563,861

(22) PCT Filed: Apr. 1, 2016
(86) PCT No.: PCT/US16/25489

§ 371 Date: Oct. 2, 2017

(51) Int. Cl. G01N 33/566; A61K 38/17; G01N 33/50; C07K 14/705

(52) U.S. Cl. CPC G01N 33/566; A61K 38/17; G01N 2500/10; C07K 14/705; G01N 2333/726; G01N 33/5041

(57) ABSTRACT

Methods of identifying agonists of MRGD for treating prediabetes, type-2 diabetes and cardiovascular diseases or disorders. Methods of treating prediabetes, type-2 diabetes and cardiovascular diseases or disorders comprising administering to a subject an agonist of MRGD.

### CROSS REFERENCE TO RELATED APPLICATIONS

This application claims the benefit of U.S. Provisional Application No. 62/142,685, filed Apr. 3, 2015, the disclosure of which is hereby incorporated by reference in its entirety.

### SEQUENCE LISTING

Incorporated by reference in its entirety is a computer readable sequence listing submitted concurrently herewith and identified as follows: “391704_ST25.txt” created on Mar. 31, 2016, at 12:32 pm, that is 4.00 KB.

### FIELD

Treatment of Diabetes and Cardiovascular Diseases and Disorders.

### BACKGROUND

Diabetes is a group of diseases characterized by defects in insulin production, insulin action, or both. The three most common forms of diabetes are type-1 diabetes, type-2 diabetes, and gestational diabetes. Type-1 diabetes, also known as insulin-dependent diabetes mellitus (IDDM), is caused by the autoimmune destruction of insulin producing pancreatic beta-cells leading to total deficiency of insulin, requiring patients with type-1 diabetes to take insulin by either injection or pump. Gestational diabetes is developed when pregnant women become intolerant to glucose. Women who have had gestational diabetes are at increased risk for developing type-2 diabetes.

Type-2 diabetes, previously known as non-insulin-dependent diabetes mellitus (NIDDM), develops as peripheral cells do not use insulin properly and then the pancreas loses its ability to produce enough insulin. Under current criteria, type-2 diabetes is diagnosed when fasting plasma glucose is ≧126 mg/dL (7.0 mmol/L); or plasma glucose level is ≧200 mg/dL (11.1 mmol/L) at 2-hours post-glucose load of 75 g; or an HbA1c (A1C) level ≧6.5% .

Prediabetes, also referred to as impaired fasting glucose (IFG) or impaired glucose tolerance (IGT), is a precursor condition to type-2 diabetes. Prediabetes is diagnosed when fasting plasma glucose is between 100 to 125 mg/dL (5.56-6.94 mmol/L); or plasma glucose level is between 140 to 199 mg/dL (7.78-11.06 mmol/L) at 2-hours post-glucose load of 75 g; or an A1C level between 5.7 and 6.4%. Without intervention and appropriate treatment, people with prediabetes are at risk for developing type-2 diabetes.

Complications of diabetes include heart disease, stroke, hypertension, blindness, other eye problems (such as diabetic retinopathy), kidney disease, nervous system disease (such as impaired sensation or pain in the feet or hands, slowed digestion of food, carpal tunnel syndrome and erectile dysfunction), amputations, periodontal disease, susceptibility to other illnesses (such as pneumonia and influenza), impaired mobility and depression. Uncontrolled diabetes can result in acute life-threatening events such as diabetic ketoacidosis and hyperosmolar coma.

In addition to lifestyle interventions, prediabetic and type-2 diabetic patients are often treated with medications to address complications of diabetes. Doctors prescribe medications to control blood pressure and blood lipids to reduce cardiovascular complications. Often, in younger and heavier patients with normal kidney function, doctors prescribe the oral drug metformin to more directly address the defects causing diabetes. Metformin suppresses hepatic glucose production, increases insulin sensitivity, enhances peripheral glucose uptake, increases fatty acid oxidation and decreases absorption of glucose from the gastrointestinal tract. Metformin, though, is contraindicated in people with any condition that could increase the risk of lactic acidosis, including kidney disorders, lung disease and liver disease. Other more recently approved drugs do not appear to be more effective than metformin and each has its own set of contraindications. For example, rosiglitazone was one of the first insulin-sensitizers used as an anti-diabetic drug, however, it is associated with an increased risk of cardiovascular events. Therefore, there is a great need for identifying additional drugs for treatment of prediabetes and diabetes.

### SUMMARY

The present invention provides a solution to this problem. The present invention is based on surprising findings that MAS-Related GPR, Member D (MRGD), a G-protein coupled receptor (GPCR), previously not linked to type-2 diabetes was found to be the target of the glucose lowering peptides DT-109 and DT-110 and that a known agonist of MRGD, β-alanine, lowers blood glucose in response to a glucose challenge.

MRGD was recently found to be a target receptor of a vasoactive peptide, alamandine. Alamandine is a newly identified component of the renin-angiotensin system (RAS) that can be formed from either angiotensin A through the action of ACE2 or from Ang-(1-7) by a decarboxylase enzyme. It has been shown that alamandine acts on MRGD to play important roles in the regulation of nociceptor function in the central nerve system and vasodilation in blood vessels with anti-hypertensive and anti-fibrotic effects. So, in addition to providing a new target for the discovery and development of additional blood glucose lowering agent, the finding that DT-109 and DT-110 are agonists of MRGD indicate that DT-109 and DT-110 may have beneficial effects on cardiovascular system.

The present disclosure provides methods of identifying candidate compounds for treating subjects having conditions associated with dysregulation of blood glucose, including, but not limited to, prediabetes and type-2 diabetes. The present disclosure also provides compounds for treating prediabetes, type-2 diabetes and cardiovascular diseases or disorders.

One embodiment of the present disclosure is a method for identifying a candidate compound for the treatment of a condition associated with a dysregulation of blood glucose. The method comprises a) providing a cell expressing MRGD; b) contacting the cell with a test compound; c) determining whether the contacting causes a response of a GPCR-mediated signaling activity of MRGD in the cell contacted with the test compound; and d) identifying the test compound as a candidate compound if the test compound causes a response of the GPCR-mediated signaling activity of MRGD.

Another embodiment of the present disclosure is a method of lowering blood glucose levels in a subject in need thereof comprising administering to the subject a composition comprising an effective amount of an MRGD agonist.

Another embodiment of the present disclosure is a method for treating a subject having a vascular or cardiovascular disease or disorder comprising administering to a subject in need thereof, a composition comprising a therapeutically effective amount of an MRGD agonist.

Still another embodiment of the present disclosure is a method for reducing the risk of cardiovascular disease or disorder comprising administering to a subject in need thereof, a composition comprising a therapeutically effective amount of an MRGD agonist.

### BRIEF DESCRIPTION OF THE FIGURES

FIG. 1A is a graph depicting blood glucose levels at various time points during an oral glucose tolerance test in C57BL/6J mice and shows that DT-109 lowers blood glucose levels in a dose dependent manner. FIG. 1B is a graph providing the data expressed as area under the curve (AUC) showing that DT-109 lowers blood glucose in a dose dependent manner.

FIG. 2 is a graphical representation of the G-protein coupled receptors and associated pathways that lead to GLP-1 release.

FIG. 3 is a graph showing that the PKA inhibitor H89 and/or the PLC inhibitor U73122 inhibit DT-110 stimulated GLP-1 secretion from a mouse enteroendocrine L cell line STC-1 cells.

FIG. 4 is a graph that shows carbachol-induced real-time calcium flux in HEK293T cells.

FIG. 5 is a graph showing the dose-response of DT-109 and DT-110 in MRGD cells

FIG. 6A is a graph showing the dose response agonist activity of DT-109 and DT-110 in GPR109a cells. FIG. 6B is a graph showing the effect of the positive control nicotinic acid in GPR109a cells.

FIG. 7A is a graph showing the dose response agonist activity of DT-109 and DT-110 in GPCR40 cells. FIG. 7B is a graph showing the effect of the positive control docosahexanoic acid in GPCR40 cells.

FIG. 8 shows the dose-response of β-alanine, DT-109, and DT-110 in MRGD cells.

FIGS. 9 A-C show the dose-response for DT-109 (FIG. 9A), DT-110 (FIG. 9B), and β-alanine (FIG. 9C) in an oral glucose tolerance test in C57BL/6 mice.

FIG. 10 is a graph showing that β-alanine stimulated calcium mobilization in MRGD cells whereas alamandine did not.

FIG. 11 is a graph showing the effect of pretreatment of MRGD cells with alamandine on calcium mobilization by DT-110, DT-109 and β-alanine.

FIG. 12 is a graph showing effect of GABA, β-alanine and DT-110 treatment on MRGD activation as measured by calcium mobilization.

FIG. 13 is a graph showing the results of an oral glucose tolerance test for GABA and DT-109.

FIG. 14 is a graph showing the results of an oral glucose tolerance test for DT-110, alamandine and metformin.

FIG. 15 is a graph showing the effect of DT-109 and alamandine on e-NOS expression.

FIG. 16 is a graph showing real-time NO release in response to DT-110 in HUVEC cells with time.

FIG. 17 is a graph showing the results of dose response study of DT-110 induced NO release from HUVEC after treatment with various concentrations of DT-110.

FIG. 18 is a graph comparing the fasting blood glucose levels in wildtype (WT) mice and MrgprD^{-/-} (KO) mice.

FIGS. 19A and 19B are graphs showing the results of oral glucose tolerance tests in wildtype (WT) and MrgprD^{-/-} (KO) mice treated with 0 or 0.5 mg/g bodyweight DT-109 (19A), or with 0 or 0.25 mg/g bodyweight DT-109 (19B). *P<0.05, KO vs. WT.

FIG. 20 is a graph showing the results of oral glucose tolerance test in wildtype (WT) and MrgprD^{-/-} (KO) mice treated with 0 or 0.5 mg/g bodyweight DT-110.

### DETAILED DESCRIPTION

“A,” “an,” “the,” “at least one,” and “one or more” are used interchangeably to indicate that at least one of the item is present; a plurality of such items may be present unless the context clearly indicates otherwise.

The terms such as “comprises”, “comprised”, “comprising”, “contains”, “containing” and the like have the meaning attributed in United States patent law; these terms are inclusive or open-ended and do not exclude additional, unrecited elements or method steps. Terms such as “consisting essentially of” and “consists essentially of” have the meaning attributed to them in United States patent law; these terms allow for the inclusion of additional ingredients or steps that do not materially affect the basic and novel characteristics of the claim invention. The terms “consists of” and “consisting of” have the meaning ascribed to them in United States patent law; these terms are close ended.

The use of the conjunction “or” is used interchangeably with at “least one of”. For example: where a composition comprises A or B, the method must comprise at least one of A and B but may also comprise both A and B. Likewise a composition comprising “A, B, C or D” must comprise at least one of the group of A, B, C and D, but may also comprise all or any combination of A, B, C and D.

Unless clearly indicated otherwise, “treatment” includes therapeutic treatment and prophylactic treatment. Therapeutic treatment is treatment of a subject that has signs or symptoms of the disease, condition or disorder to be treated. Prophylactic treatment refers to treatment of a subject that is predisposed to the disease, condition or disorder that does not show overt signs of the disease, condition or disorder.

DT-109 refers to the three amino acid peptide glycine-glycine-leucine (GGL) which has the structural formula:

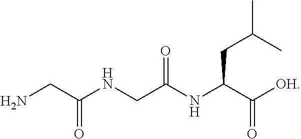 [SMILES: CC(C)C[C@H](NC(=O)CNC(=O)CN)C(=O)O]

DT-110 refers to the tripeptide glycine-glycine-D-leucine (GGdL), where dL is the D-stereoisomer of leucine. GGdL has the structural formula:

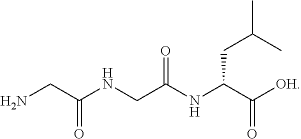 [SMILES: CC(C)C[C@@H](NC(=O)CNC(=O)CN)C(=O)O]

GABA is an abbreviation for gamma amino butyric acid.

MRGD is an abbreviation for MAS-related G-protein-coupled receptor member D. MRGD. The gene encoding MRGD is referred to as MRGPRD or MrgprD.

GPCR is an abbreviation for G-protein coupled receptor.

Plasma glucose and blood glucose are used interchangeably herein.

The term “therapeutically effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to diabetes, a non-limiting example is an amount that sufficient to lower blood glucose.

As used herein the term “subject” refers to a mammal, e.g. a human.

FLIPR is an abbreviation for Fluorescent Imaging Plate Reader.

Test compounds suitable for use in the assays of the present disclosure include, but are not limited to, amino acids, peptides, antibodies or fragments thereof, and small molecule compounds.

One aspect of the present disclosure provides method of screening to identify candidate compounds for treating a condition associated with high blood glucose levels. The condition associated with high blood glucose is for example prediabetes or type-2 diabetes.

Identification of candidate compounds using methods of the invention reduces the number of compounds that need to be screened by more specific in vitro or in vivo assays such as measurement of GLP-1 secretion, insulin secretion, glucose tolerance, blood glucose levels, blood pressure, and blood level of nitric oxide.

One embodiment of the present disclosure is a method for identifying a candidate compound for the treatment of a condition associated with a dysregulation of blood glucose. The method comprises a) providing a cell expressing MRGD; b) contacting the cell with a test compound; c) determining whether the contacting causes a response of a GPCR-mediated signaling activity of MRGD in the cell contacted with the test compound; and d) identifying the test compound as a candidate compound if the test compound causes a response of the GPCR-mediated signaling activity of MRGD.

Another embodiment of the present disclosure is a method for identifying a candidate compound for the treatment of a condition associated with a cardiovascular disease or disorder. The method comprises a) providing a cell expressing MRGD; b) contacting the cell with a test compound; c) determining whether the contacting causes a response of a GPCR-mediated signaling activity of MRGD in the cell contacted with the test compound; and d) identifying the test compound as a candidate compound if the test compound causes a response of the GPCR-mediated signaling activity of MRGD.

In various embodiments the response of the GPCR-mediated signaling activity of MRGD is an increase in the concentration of intracellular cyclic adenosine monophosphate (cAMP), an increase in the concentration of intracellular inositol trisphosphate (IP3), or an increase in the concentration of intracellular calcium, or any combination thereof.

In various embodiments of the first aspect of the present disclosure, the candidate compound is further tested to provide additional information regarding the candidate target which may result in the compound being further developed or discontinued. Embodiments of the present disclosure is direct to providing such additional information. One embodiment is a method for identifying a candidate compound for the treatment of a condition associated with a dysregulation of blood glucose. The method comprises a) providing a cell expressing MRGD; b) contacting the cell with a test compound; c) determining whether the contacting causes a response of a GPCR-mediated signaling activity of MRGD in the cell contacted with the test compound; and d) identifying the test compound as a candidate compound if the test compound causes a response of the GPCR-mediated signaling activity of MRGD, further comprising determining if the candidate compound stimulates GLP-1 secretion by an enteroendocrine L-cell. In a particular embodiment the enteroendocrine L-cell is an STC-1 cell.

To further evaluate a test or candidate compound it is desirable to test the compound in an in vivo systems to determine the candidate compound lowers blood glucose. The present invention provides in vivo methods to provide such additional information. One embodiment of the present disclosure is a method for identifying a candidate compound for the treatment of a condition associated with a dysregulation of blood glucose. The method comprises a) providing a cell expressing MRGD; b) contacting the cell with a test compound; c) determining whether the contacting causes a response of a GPCR-mediated signaling activity of MRGD in the cell contacted with the test compound; and d) identifying the test compound as a candidate compound if the test compound causes a response of the GPCR-mediated signaling activity of MRGD, further comprising administering the candidate compound to an MRGD wildtype mouse, and determining if the candidate compound lowers blood glucose levels.

Further it may be desirable to determine if the compound acts, partially acts or does not act through a particular pathway. Some compounds may show little or no glucose lowering activity in the MRGD knockout mouse compared with a control mouse (wildtype mouse of the same species from which the knockout mouse was created) and these compounds would be considered to act through MRGD. Some compounds may show significant blood glucose lowering activity in control mice and show less, yet significant, glucose lowering activity in the MRGD knockout mouse. These compounds may be partial agonist of MRGD or may work through a different mechanism. Some compounds may show significant blood glucose lowering activity in the MRGD knockout mouse, similar to or greater than in the control mouse, these compounds would be considered to act independently of MRGD. The present disclosure provides method of evaluating compounds to determine if a compound acts through MGRD. The compound may be a test compound or a candidate compound identified by a method of the present disclosure. In particular the disclosure provides methods for determining if a compound lowers blood glucose in vivo and if the compounds acts through MGRD.

One embodiment of the present disclosure is a method of determining if compound acts through MRGD, the method comprising: providing a MRGD knockout mouse; administering the compound to the knockout mouse; providing a wildtype mouse control mouse; administering the compound to the wildtype control mouse; determining if administration of the compound lowers blood glucose levels in the knockout mouse; determining if administration of the compound lowers blood glucose levels in the knockout mouse, wherein if the compound does not significantly lower blood glucose in the knockout mouse or if the compound lowers blood glucose levels significantly less than in the control mouse, the compound is determined to act at least partially through MRGD.

A second aspect of the present disclosure provides methods for the treatment of diabetes and prediabetes. One embodiment of the present disclosure is a method of lowering blood glucose levels in a subject in need thereof comprising administering to the subject a composition comprising an effective amount of an MRGD agonist. Another embodiment is a method of lowering blood glucose levels in a subject in need thereof, comprising administering to the subject a composition comprising an effective amount of an MRGD agonist, wherein the MRGD agonist is not β-alanine, GABA, GGL (DT-109), GLG, GLL, LLG, LGL, LGG, GGdL (DT-110), GdLG, GdLL, GLdL, GdLdL, dLLG, LdLG, dLdLG, dLGG, dLGL, LGdL, or dLGdL.

Yet another embodiment is a method of reducing HbA1c levels in a subject in need thereof, comprising administering to the subject a composition comprising an effective amount of an MRGD agonist. Still another embodiment is a method of reducing HbA1c levels in a subject in need thereof, comprising administering to the subject a composition comprising an effective amount of an MRGD agonist, wherein the MRGD agonist is not β-alanine, GABA, GGL (DT-109), GLG, GLL, LLG, LGL, LGG, GGdL (DT-110), GdLG, GdLL, GLdL, GdLdL, dLLG, LdLG, dLdLG, dLGG, dLGL, LGdL, or dLGdL.

Another embodiment of the present disclosure is a method of treating a subject having prediabetes comprising administering to the subject a composition comprising an effective amount of an MRGD agonist. Still another embodiment of the present disclosure is a method of treating a subject having prediabetes comprising administering to the subject a composition comprising an effective amount of an MRGD agonist, wherein the MRGD agonist is not β-alanine, GABA, GGL (DT-109), GLG, GLL, LLG, LGL, LGG, GGdL (DT-110), GdLG, GdLL, GLdL, GdLdL, dLLG, LdLG, dLdLG, dLGG, dLGL, LGdL, or dLGdL.

Another embodiment of the present disclosure is a method of treating a subject having type-2 diabetes comprising administering to the subject an effective amount of a composition comprising an MRGD agonist. Still another embodiment of the present disclosure is a method of treating a subject having type-2 diabetes comprising administering to the subject an effective amount of a composition comprising an MRGD agonist, wherein the MRGD agonist is not β-alanine, GABA, GGL (DT-109), GLG, GLL, LLG, LGL, LGG, GGdL (DT-110), GdLG, GdLL, GLdL, GdLdL, dLLG, LdLG, dLdLG, dLGG, dLGL, LGdL, or dLGdL. Yet another embodiment of the present disclosure is a method of treating a subject having type-2 diabetes comprising administering to the subject an effective amount of a composition comprising an MRGD agonist, wherein the MRGD agonist is not β-alanine, GABA, GGL (DT-109), GLG, GLL, LLG, LGL, LGG, GGdL (DT-110), GdLG, GdLL, GLdL, GdLdL, dLLG, LdLG, dLdLG, dLGG, dLGL, LGdL, or dLGdL

Another embodiment of the present disclosure is a method for increasing GLP-1 levels in a subject comprising administering to the subject an effective amount of a composition comprising an MRGD agonist. Yet another embodiment of the present disclosure is a method for increasing GLP-1 levels in a subject comprising administering to the subject an effective amount of a composition comprising an MRGD agonist, wherein the MRGD agonist is not β-alanine, GABA, GGL (DT-109), GLG, GLL, LLG, LGL, LGG, GGdL (DT-110), GdLG, GdLL, GLdL, GdLdL, dLLG, LdLG, dLdLG, dLGG, dLGL, LGdL, or dLGdL.

Another embodiment of the present disclosure is a method for increasing the secretion of insulin comprising administering to a subject a composition comprising an effective amount of MRGD agonist. Still another embodiment of the present disclosure is a method for increasing the secretion of insulin comprising administering to a subject a composition comprising an effective amount of MRGD agonist, wherein the MRGD agonist is not β-alanine, GABA, GGL (DT-109), GLG, GLL, LLG, LGL, LGG, GGdL (DT-110), GdLG, GdLL, GLdL, GdLdL, dLLG, LdLG, dLdLG, dLGG, dLGL, LGdL, or dLGdL.

A third aspect of the present disclosure provides methods for treating cardiovascular disorders or reducing the risk of developing cardiovascular disease or cardiovascular disorder.

One embodiment of the present disclosure is a method for treating a subject having a cardiovascular disease or disorder comprising administering to a subject in need thereof, a composition comprising a therapeutically effective amount of an MRGD agonist. In a particular embodiment of the embodiment above, the MRGD agonist is DT-109, DT-110 or a combination thereof. In another embodiment of the methods for treating cardiovascular disorders or reducing the risk of developing cardiovascular disease or cardiovascular disorder the MRGD agonist is not alamandine.

One embodiment of the present disclosure is a method for reducing the risk of cardiovascular disease or disorder comprising administering to a subject in need thereof, a composition comprising a therapeutically effective amount of an MRGD agonist. In a particular embodiment of the embodiment above, the MRGD agonist is DT-109, DT-110 or a combination thereof.

In various embodiments of the methods of the present disclosure, the cardiovascular disease or disorder is hypertension, acute coronary syndrome, angina, heart failure, or a lipid disorder.

Another embodiment of the present disclosure is a method for increasing the expression of eNOS in a subject in need thereof, a composition comprising a therapeutically effective amount of an MRGD agonist. In a particular embodiment of the embodiment above, the MRGD agonist is DT-109, DT-110 or a combination thereof. In another embodiment the method for increasing the expression of eNOS, the MRGD agonist is not alamandine.

Yet another embodiment is a method for increasing the blood level nitric oxide in a subject comprising administering to the subject a composition comprising a therapeutically effective amount of an MRGD agonist. In a particular embodiment of the embodiment above, the MRGD agonist is DT-109, DT-110 or a combination thereof. In another embodiment of the method for increasing the blood level of nitric oxide, the MRGD agonist is not alamandine.

Still another embodiment is a method for lowering blood pressure comprising administering to the subject in need thereof, a composition comprising a therapeutically effective amount of an MRGD agonist. In a particular embodiment of the embodiment above, the MRGD agonist is DT-109, DT-110 or a combination thereof. In another embodiment of a method for lowering blood pressure the MRGD agonist is not alamandine.

Based on the ability of MRGD agonists to stimulate GLP-1 secretion, MRGD agonists may be effective for reducing weight in a subject. Therefore, another embodiment of the present disclosure is a method reducing weight in a subject comprising administering to the subject a composition comprising a therapeutically effective amount of an MRGD agonist. In a particular embodiment of the embodiment above, the MRGD agonist is DT-109, DT-110 or a combination thereof.

Cell lines that stably overexpress MRGD are available commercially or may be generated by methods known in the art (see e.g., Ajit, S. K., et al, J Biomed Biotechnol. 2010: 326020. published online Sep. 14, 2010, and Zhang R., et al., Acta Pharmacol Sin (2007) vol 28(1): 125-131). The MRGD may be human MRGD or an ortholog MRGD such as rat, mouse, or non-human primate. Genebank accession numbers for the protein sequence and amino acid sequence of the human ortholog of MRGD are NP944605.2 and NM_198923.2, respectively.

Pharmaceutical compositions of the present disclosure are formulated with pharmaceutically acceptable excipients such as carriers, solvents, stabilizers, diluents, etc., depending upon the particular mode of administration and dosage form. Pharmaceutical compositions of the present disclosure may be administered to a subject, such as a human subject, in a variety of forms adapted to the chosen route of administration, i.e., orally, ocular, intradermal, intranasal, transdermal, and parenterally, by intravenous, intramuscular or subcutaneous routes. Such compositions, forms, and methods for their preparation are well known and may be found, for example, in Remington's Pharmaceutical Sciences, 19th Edition (Mack Publishing Company, 1995).

MRGD agonists of the present invention may be administered alone, as a monotherapy, or in combination with an additional agent. Such combination therapies include, but are not limited to simultaneous or sequential administration of the compound of the present invention and the additional agent. Monotherapy, in the context of the present disclosure, means that the MRGD agonist is administered as the sole active ingredient for treating the underlying disease or disorder.

### EXAMPLES

### Example 1

### Effect of DT-109 on Blood Glucose Levels

Previous studies have shown that DT-109 and DT-110 lower blood glucose in C57/BL/6J mice and diabetic mouse models. (See Zhang J, et al., PLoS One 2013; vol.8, Issue 12, published online e83509 and U.S. Pat. No. 8,664,177).

The effect of DT-109 dosage on blood glucose levels of C57BL/6J mice was further determined in an oral glucose tolerance test (OGTT). C57BL/6J mice were received from Jackson Laboratories at 4 weeks old and aged to 7 weeks and then grouped by body weight into 4 groups of 10. The mice were fasted overnight (12 hours) prior to the assay. Body weights and blood glucose (FreeStyle Lite glucometer) levels were determined. Immediately following baseline glucose measurements the groups were dosed with 2 mg/g glucose and vehicle (PBS 10×), or 2 mg/g glucose and a DT-109 dose concentration of 1.0, 0.5, or 0.1 mg/g body weight. One bolus oral dose (gavage) delivered the glucose and the group specific concentration of DT-109. At 30, 60, 90, and 120 minutes glucose levels were measured using whole blood collected from the tip of the tail via needle puncture. Upon the final 120 min measurement, food was returned to the mice.

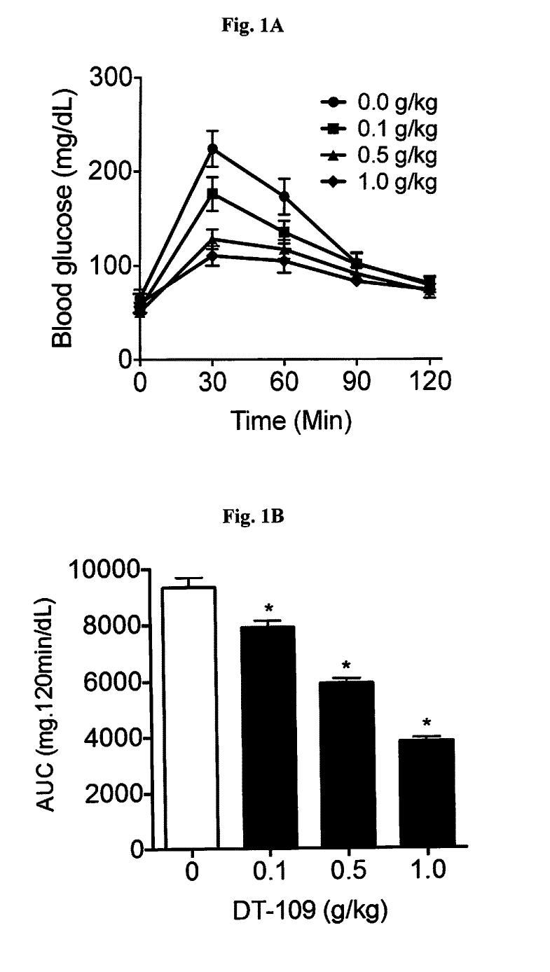

As shown in FIG. 1, DT-109 lowers blood glucose levels in C57BL/6J mice during OGTT in a dose dependent manner (FIG. 1A). Analysis of the area under curve (AUC) for each group clearly showed that DT-109 significantly lowers blood glucose in mice in a dose dependent manner (FIG. 1B, * p<0.01). Significance was calculated relative to control using one-way ANOVA in GraphPad PRISM software.

### Example 2

### DT-110 Acts Through GPCR Signaling to Elicit its Activity

It is known that luminal protein hydrolysates (peptone) increase the levels of several gut hormones including GLP-1 through a GPCR-mediated signaling pathway. Because DT-109 has been shown to elevate GLP-1 level in diabetic mice and stimulate GLP-1 secretion in an enteroendocrine L-cell line (STC-1), we examined whether or not GPCRs were involved in DT-110 stimulated GLP-1 secretion.

GPCRs comprise a large protein family of 7 transmembrane receptors and are targets of approximately 30-40% of currently marketed drugs. There are three main G-protein-mediated signaling pathways, mediated by four sub-classes of G-proteins (G_{αs}, G_{αi/o}, G_{αq/11}, and G_{α12/13}) (37). The effector of both the G_{αs} and G_{αi/o} pathways is adenylate cyclase (AC) that catalyzes the conversion of ATP to cAMP, of which elevation further activates the cAMP-dependent protein kinase A (PKA) family. The effector of the G_{αq/11} pathway is phospholipase C-β (PLC-β) that catalyzes the cleavage of membrane-bound phosphatidylinositol 4,5-biphosphate (PIP2) into the second messenger inositol (1,4,5) trisphosphate (IP3). IP3 elicits Ca²⁺ release from endoplasmic reticulum (E.R.) and diacylglycerol (DAG) that activates protein kinase C (PKC).

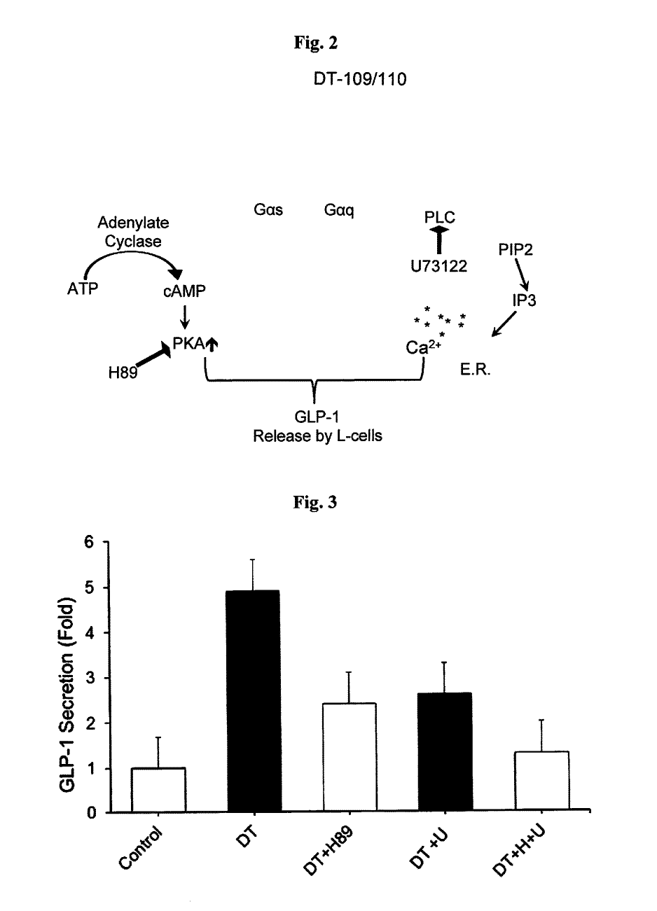

To examine the involvement of GPCR signaling pathway in DT-110 action, we examined the role of PKA and PLC in DT-110-stimulated GLP-1 secretion in STC-1 cells (endocrine L-cell line) using H89 and U73122 which are specific inhibitors of PKA and PLC respectively. The pathways and sites of action of H89 and U73122 are graphically represented in FIG. 2.

The mouse STC-1 cells (enteroendocrine L-cell line) were obtained from ATCC (accession number CRL-3254) For determination of the involvement of GPCR signaling pathway in DT-110 action, STC-1 cells were cultured in DMEM medium with 10% horse serum and 2% FBS. At confluence of 80%, cells were treated with DT-110, DT-110+PLC inhibitor U73122, DT-110+PKA inhibitor H89, or DT-110+U73122+H89 in DMEM medium (without serum and glucose) for 2 hours. Supernatants were collected for GLP-1 measurement. and the GLP-1 concentration was measured using a total GLP-1 (7-36 and 9-36) ELISA kit according to manufacturer's instruction (Alpco. Salem, N.H.).

The resulting data show that DT-110 stimulated significant release of GLP-1 by STC-1 cells, which was partially inhibited by either U73122 or H89, and essentially abolished by U73122+H89, indicating that a GPCR-mediated signaling pathway is involved in DT-110 action. Results are shown in FIG. 3; DT is DT-110, H is H89, and U is U73122. These results suggest that DT-110 acts via one or more GPCR to trigger GLP release.

### Example 3

### Identification of GPCR Target for DT-109 and DT-110

To identify the specific GPCR target(s), a high throughput screening of GPCR library was performed with DT-110 using the EMD Millipore GPCR Profiler Service, a complete cell-based functional platform that uses a common validated readout. The platform uses ChemiScreen GPCR stable cell lines that are used for real-time calcium flux assays to rapidly, screen a spectrum of GPCRs. DT-110 was used to screen a library with 173 cell lines, each over expressing a different GPCR. Percentage activation values were determined upon initial addition of compound(s). Percentage activation and inhibition values were determined for each GPCR relative to the reference compounds.

Assays were performed according to the manufacturer's instructions. Briefly, DT-110, vehicle controls and positive controls were prepared in the Millipore's GPCRProfiler® Assay Buffer according to the Millipore's protocol. For the agonist assay, DT-110 was plated in duplicate for each concentration assayed. Reference agonist at ECmax for each GPCR assayed was prepared in a similar manner to serve as assay control. The assay plate was read by the FLIPR TETRA® (Molecular Devices, Sunnyvale, Calif.). All assay plate data were subjected to appropriate baseline corrections and the maximum fluorescence values were processed to calculate percentage activation (relative to ECmax reference agonist and vehicle control values). Three GPCRs were activated by DT-110 at a concentration of 12.5 mM. These were GPCR40, the free fatty acid receptor, GPCR 109a, the nicotinic acid receptor, and Mas-Related GPCR D (MRGD) with mean percentage activation values of 50.2%, 54.3%, and 64.5%, respectively. Of these 3 receptors, only MRGD was activated at both 12.5 and 1.25 mM DT-110 (Table 1). Interestingly, the 3 GPCRs identified are expressed in enteroendocrine L-cells and pancreatic β-cells.

TABLE 1

| GPCR | DT-110 @ 12.5 mM* | | | DT-110 @ 1.25 mM* | | |
|---|---|---|---|---|---|---|
| Target | n1 | n2 | Avg. | n1 | n2 | Avg. |
| GPCR40 | 55.9 | 44.5 | 50.2 | −9.3 | −9.6 | −9.5 |
| GPCR109a | 52.9 | 55.7 | 54.3 | −8.1 | −8.0 | −8.1 |
| MC2 | −1.0 | −0.9 | −1.0 | 0.6 | −2.0 | −0.7 |
| MC4 | −0.2 | 7.0 | 3.4 | 3.0 | −4.0 | −0.5 |
| MC5 | −0.6 | 0.0 | −0.3 | 0.7 | −0.8 | 0.0 |
| MCHR1 | −0.5 | 0.3 | −0.1 | 0.2 | 0.7 | 0.4 |
| MCHR2 | −0.8 | −0.9 | −0.9 | 0.1 | 0.8 | .5 |
| Motilin | −0.3 | −0.5 | −0.4 | −0.3 | −0.3 | −0.3 |
| MRGD | 60.7 | 68.3 | 64.5 | 76.5 | 62.0 | 69.2 |
| MRGX1 | −6.7 | −6.6 | −6.6 | −1.3 | −2.5 | −1.9 |
| MRGX2 | 0.4 | −0.4 | 0.0 | 0.0 | −0.4 | −0.2 |
| NK1 | −1.0 | −1.1 | −1.1 | −1.0 | −0.9 | −0.9 |
| NK2 | −9.5 | −9.7 | −9.6 | −9.4 | −9.6 | −9.5 |
| NMUR1 | −0.7 | 0.5 | −0.1 | 1.3 | −0.4 | 0.5 |
| NMUR2 | 0.4 | −2.2 | −0.9 | 0.9 | 3.9 | 2.4 |
| NOP | 3.4 | −8.0 | −2.3 | −7.8 | 7.6 | −0.1 |
| NPBW1 | −0.8 | −0.9 | −0.9 | 1.6 | −1.1 | 0.2 |

*Numbers are the percent activation compared to standards.

### Example 4

### GPCR Assays Using GPR 40, GPR 109A and MRGD Cell Lines

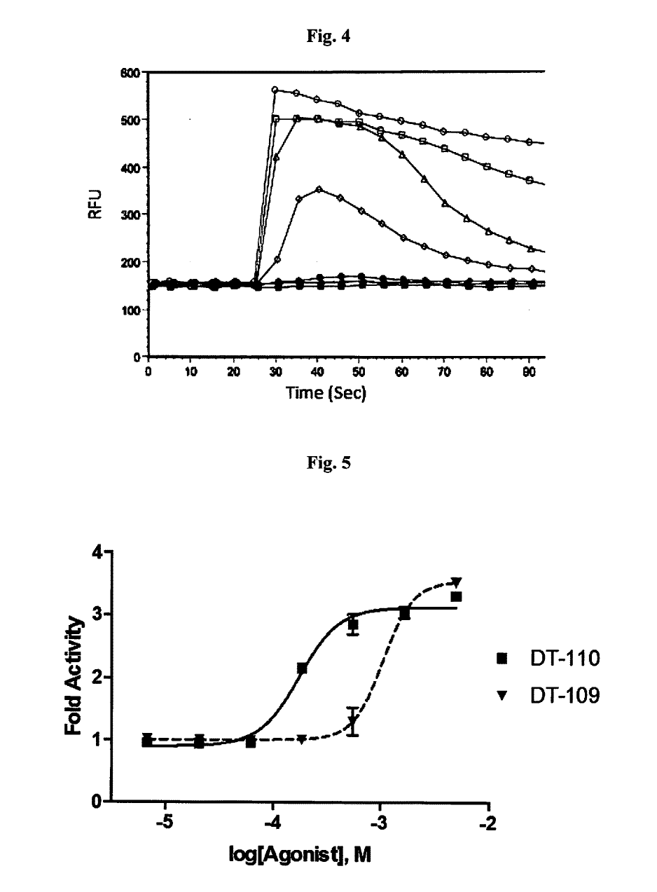

Real-time Ca2+ flux assays. To further validate the GPCRProfiler results, we established a real-time Ca2+ flux assay using a FlexStation 3 Microplate Reader (Molecular Devices). Fluorescent Ca2+ indicators are widely used for in-cell measurement of agonist-stimulated and antagonist-inhibited calcium signaling through GPCRs. The Fluo-4 Direct™ Calcium Assay Kits (Life Technologies), which allows direct addition to wells containing cells growing in culture media without the requirement of media removal or a wash step, is used to monitor Ca2+ flux in cells according to the manufacturer's protocol. Briefly, HEK293T cells are plated and cultured in 96-well microplates to near confluence. The microplates containing cells are removed from the incubator and an equal volume of 2× Fluo-4 Direct™ calcium reagent loading solution is added directly to wells containing cells in culture medium. The plates are incubated for 30 minutes at 37° C. and 30 minutes at room temperature. Then the plates (no need to remove the medium or Fluo-4 Direct™ calcium reagent) are placed on FlexStation 3 with settings appropriate for excitation at 494 nm and emission at 516 nm. FlexStation 3 can directly transfer reagents from 96-well source plates to a read plate one column at a time. After automatic loading of various concentrations of DT-110, real-time Ca²⁺ flux is recorded for up to 3 minutes and the peak value of Ca2+ flux represents maximum stimulation at the concentration used. FIG. 4 shows that higher concentrations of DT-110 stimulate greater Ca²⁺ flux. The various concentrations of DT-110 (mM) are represented in the graph as follows: open circles, 0.1; open squares, 0.033; open triangles, 0.011; and open diamonds 0.0037. Shaded shapes are negative controls.

In order to further investigate the activity of DT-109 and DT-110, ChemiScreen™ GPR 40, GPR 109A and MRGD over-expressing cell lines HEK 293T cells (Chem-1, EMD Millipore) were purchased which overexpress high levels of endogenous Gα15 and are stably transfected with the recombinant human GPCRs. Cells were grown in DMEM medium containing 10% FBS, 1× non-essential amino acids (Gibco #111450-040), 20 mM HEPES and 250 μg/mL Geneticin® (Gibco #10131-035). Cells were harvested with 0.25% Trypsin/EDTA (Gibco #25200-056), pelleted at 1,200×g for 6 minutes and resuspended in DMEM media described above minus Geneticin®. The cells were seeded at 4×10⁴ cells/well/100 μL of a clear bottom/black 96-well plate and grown overnight. The calcium mobilization assays was performed using Screen Quest Fluo-8 Medium Removal Calcium Assay Kit (AAT Bioquest #36308) according to the manufacturer's protocol. Briefly, cells were washed with 100 μL volume of Hank's balanced salt solution, 20 mM HEPES, pH 7.4 and loaded with a buffer containing HBSS, 20 mM HEPES, 1× Pluronic F127 and the Fluo-8 NW dye. The cells were incubated for 30 minutes at 37° C. followed by a 30 minute room temperature incubation. Drug stocks were prepared at 100× concentration and diluted 1:33 in HBSS, 20 mM HEPES, pH 7.4 in a 96-well plate (Corning #3641). Calcium mobilization was measured using the FlexStation 3 (Molecular Devices) at excitation wavelength of 490 nm and emission wavelength of 525 nm. Fluorescence was measured for 180 seconds at 5 second intervals with the addition of compound at a 1:3 dilution after 15 seconds. Peak fluorescence values were calculated using SoftMax Pro 5 (Molecular Devices). The fluorescence values obtained from control wells without cells are subtracted from wells containing treated cells to correct for background. The ratio of fluorescence values of treated cells to vehicle control wells was used to calculate fold activity of the compounds. As expected both DT-109 and DT-110 were active at 5 mM, the highest dose used in this study. DT-110 had an EC50 of 76 μM and DT-109 had an EC50 of 600 μM. Representative results are shown in FIG. 5.

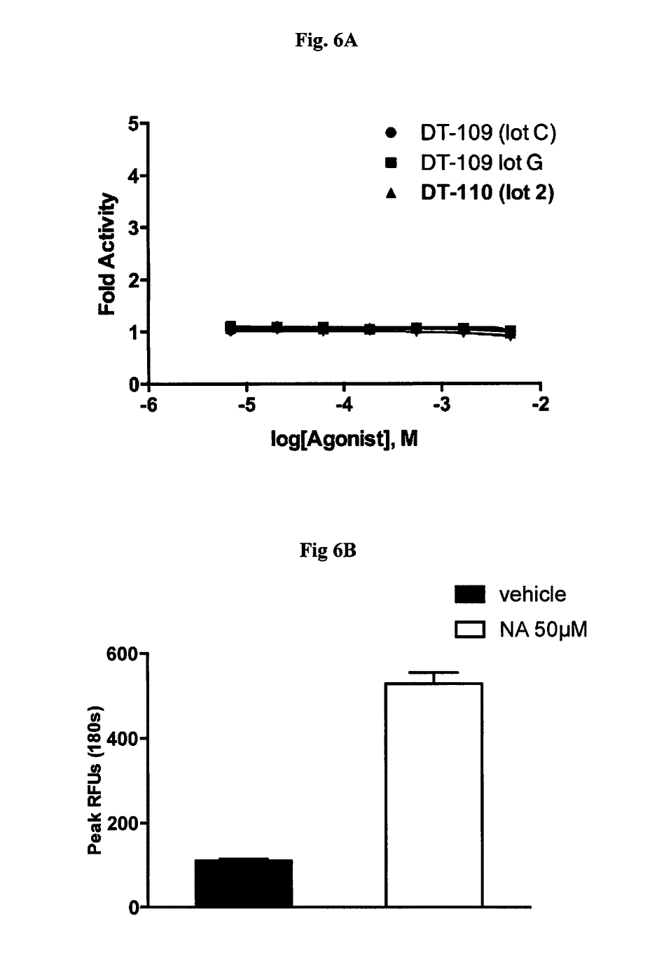

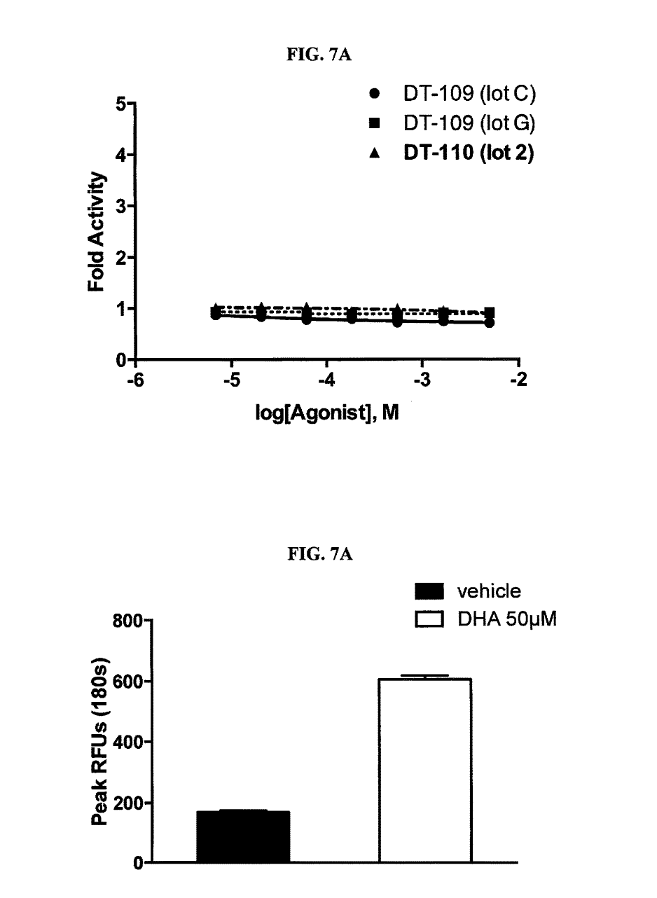

The specificity of the agonist activity was determined by testing in the cell lines that over-express GPCR40 and GPCR 109a. FIG. 6A shows the dose response effect of DT-109 (two different lots) and DT-110 in GPR109 cells as measured by Ca²⁺ Flux. FIG. 6B shows results for the positive control nicotinic acid (NA) at 50 μM. FIG. 7A shows the dose response effect of DT-109 (two different lots) and DT-110 in GPCR40 cells. FIG. 7B shows results for the positive control docosahexanoic acid (DHA). It can be seen that DT-109 and DT-110 are inactive in on GPCR 109a and GPCR 40 at doses of 5 mM and below. These results indicate that DT-109 and DT-110 act through the MRGD and not GPCR 40 or GPCR 109a receptors.

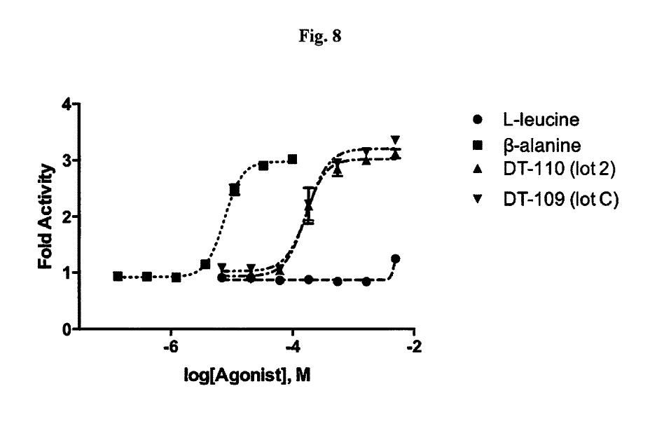

In contrast, as shown in FIG. 8, DT-109 and DT-110 act through MRGD. β-alanine was used as a positive control for activity in the MRGD cell line. The EC50 of β-alanine was determined in an assay that also examined the activity of DT-110, DT-109, and a negative control (L-leucine). As shown in FIG. 8 the EC50 of β-alanine was 7.5 μM, DT-110 (lot 2) was 161.2 μM and DT-109 was 177.8 μM in this cell-based assay. β-alanine is more than 10-fold more potent than DT-110 or DT-109 on this receptor. These results suggest that β-alanine might also be effective at lowering blood glucose in vivo.

### Example 5

### Oral Glucose Tolerance Test of DT-109, DT-110 and β-Alanine

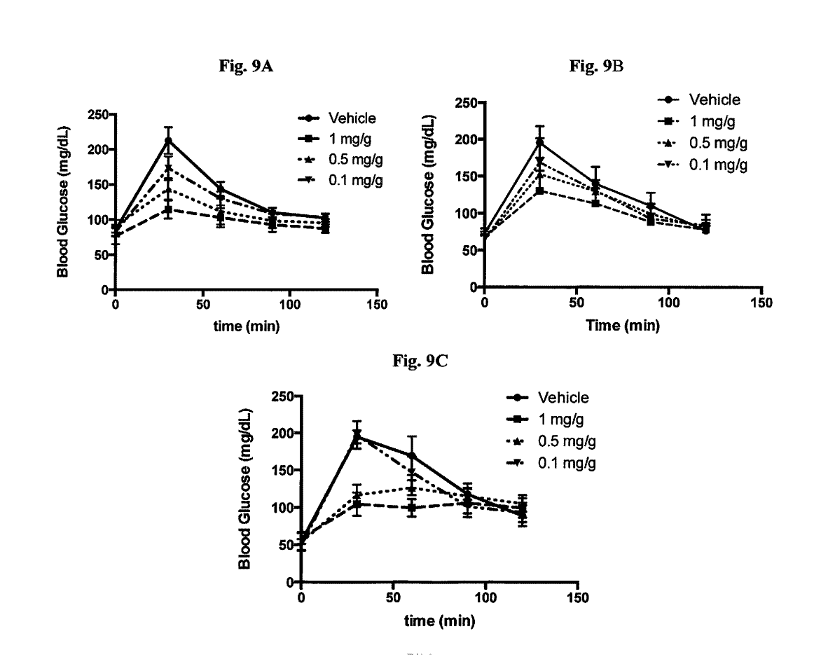

Oral glucose tolerance tests (OGTTs) were performed as described in Example 1. OGTTs in were carried out in C57BL/6J mice for DT-109, DT-110 and β-alanine at 0 (vehicle), 1, 0.5 and 0.1 mg/g body weight (n=10 for each group) . At 0, 30, 60, 90, and 120 minutes glucose levels were measured using whole blood collected from the tip of the tail via needle puncture These results show that DT-109, DT-110 and β-alanine lower blood glucose in response to a glucose load, in a dose-dependent manner. FIGS. 9 A-C show the OGTT dose response for DT-109 (A), DT-110 (B), and β-alanine (C). In FIG. 9A (DT-109) all treatments are significant (P<0.0001). In FIG. 9B (DT-110), treatments are significant for 1 mg/g (P<0.0001) 0.5 mg/g (P=0.01 to 0.001) and 0.1 mg/g (P<0.05). In C (β-alanine), treatments with 1.0 and 0.5 mg/g showed significance (P<0.0001) while treatment with 0.1 mg/g did not. It can be seen in all three figures that the hierarchy of response is 1 mg/g>0.5 mg/g>0.1 mg/g. This same hierarchy is also seen in the Ca²⁺ flux assay using the MRGD cell line.

### Example 6

### Alamandine Binds to MRGD and Acts as an Antagonist of β-Alanine

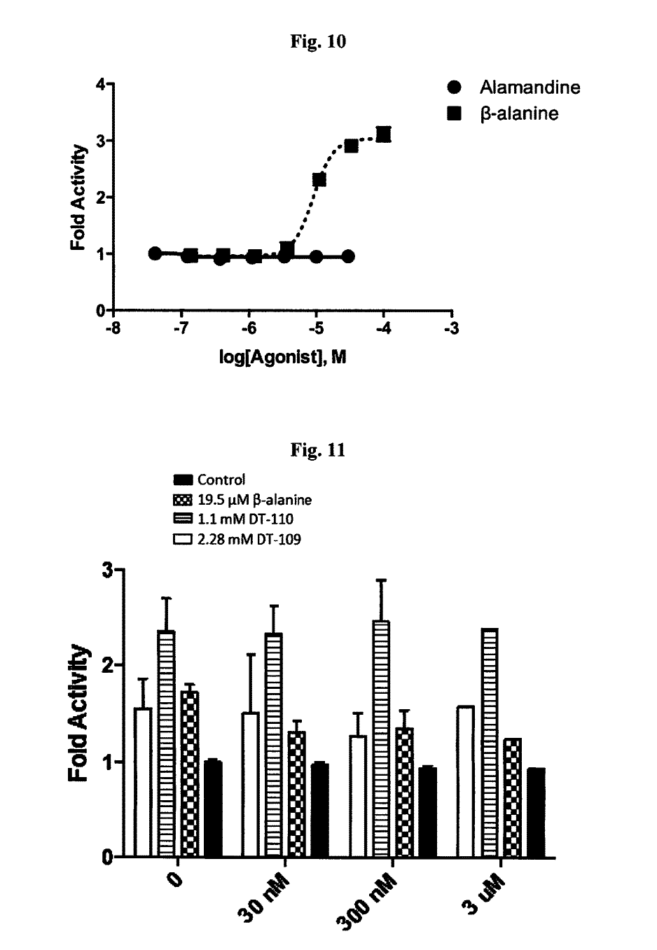

MRGD cells (EMD Millipore, MA) were treated with either β-alanine, or alamandine at various concentrations (FIG. 10) and calcium mobilization was monitored using a FlexStation 3 Microplate Reader (Molecular Devices) as described in Example 4. As shown in FIG. 10, the agonist activity of β-alanine is between approximately 5 μM and 50 μM with a half maximal effective concentration (EC₅₀) of about 9 μM. Alamandine showed no activation of calcium mobilization over the entire dose range.

A similar assay was run in the antagonist mode where cells were first incubated with 0, 30 nM, 300 nM or 3 μM alamandine as shown in FIG. 11 (x-axis) for 15 minutes. Then one of 3 different agonists was added to the wells, at their EC90 and the calcium mobilization recorded. As shown in FIG. 11, alamandine significantly decreased β-alanine signaling in this cell line (P<0.05, one-way ANOVA). Surprisingly, pre incubation with alamandine did not have any significant effect on DT-110 or DT-109 signaling. These results indicate that alamandine can act as an antagonist to a known agonist of MRGD. In addition, because DT-110 and DT-109 are agonists of MRGD, these results suggest that alamandine and β-alanine interact with MRGD at a site that is different from the site at which DT-109 and DT-110 interact.

### Example 7

### Activation of MRGD Cells by β-Alanine, GABA, and DT-110

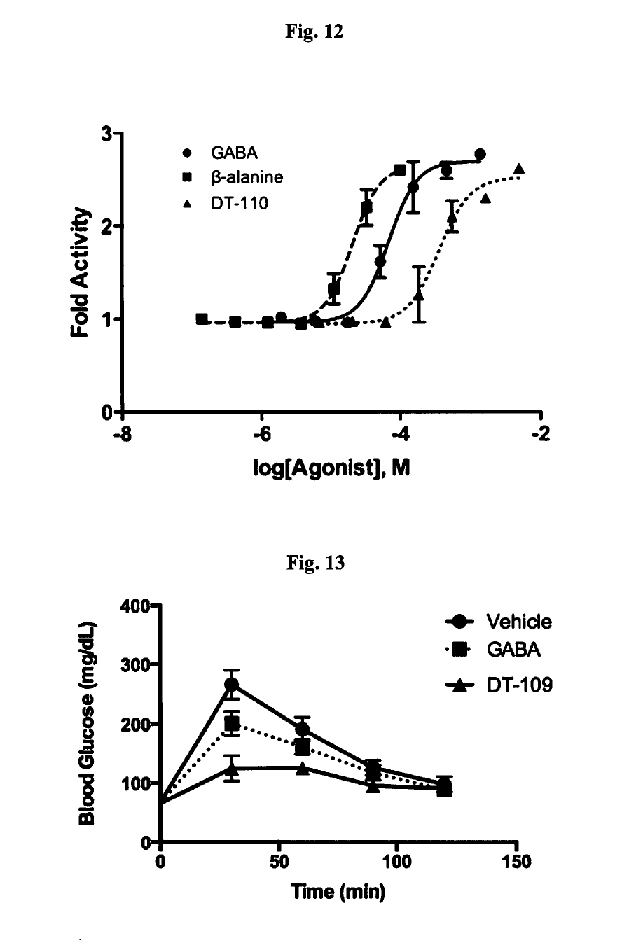

MRGD cells (EMD Millipore, MA) were treated with GABA, β-alanine, or DT-110 at various concentrations and calcium mobilization was monitored using a FlexStation 3 Microplate Reader (Molecular Devices) as described in Example 4. As shown in FIG. 12, the agonist activity of β-alanine, GABA, and DT-110 have half maximal effective concentration (EC₅₀) of about 21 μM, 68 μM, and 360 μM, respectively.

### Example 8

### Oral Glucose Tolerance Test of GABA and DT-109

C57BL/6J mice were received from Jackson Laboratories at 4 weeks old and aged to 7 weeks and then grouped by body weight into 4 groups of 10. The mice were fasted overnight (12 hours) prior to the assay. Body weights and blood glucose (FreeStyle Lite glucometer) levels were determined. Immediately following baseline glucose measurements the groups were dosed with 2 mg/g glucose and vehicle (PBS 10×), GABA (200 mg/kg) or DT-109 (1 mg/g). At 30, 60, 90, and 120 minutes glucose levels were measured using whole blood collected from the tip of the tail via needle puncture. As shown in FIG. 13, both GABA and DT-109 had an overall significant effect in lowering blood glucose (p<0.01 2-way ANOVA).

### Example 9

### Oral Glucose Tolerance Test of DT-110, Alamandine and Metformin

C57BL/6J mice were received from Jackson Laboratories at 4 weeks old and aged to 7 weeks and then grouped by body weight into 4 groups of 10. The mice were fasted overnight (12 hours) prior to the assay. Body weights and blood glucose (FreeStyle Lite glucometer) levels were determined. Immediately following baseline glucose measurements the groups were dosed with 2 mg/g glucose and vehicle (PBS 10×), DT-110 (1 mg/g), alamandine (10 mg/g) or metformin (0.2 mg/g). At 30, 60, 90, and 120 minutes glucose levels were measured using whole blood collected from the tip of the tail via needle puncture.

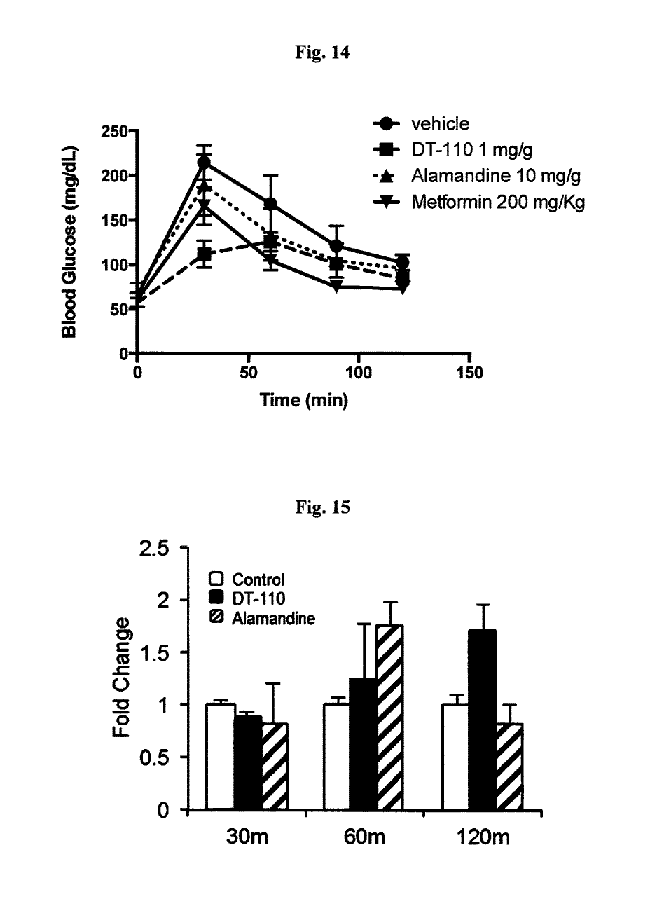

In the vehicle group, blood glucose was raised from 65 to 215 mg/dL by the glucose challenge. After 120 minutes, blood glucose had dropped to 102 mg/dL. Alamandine treatment had a weak but overall significant effect (P=0.01 to 0.001 2-way ANOVA) in lowering blood glucose compared to vehicle control levels. As shown in FIG. 14, the overall effect of DT-110 was significant (P<0.001) and dramatically blocked the rise in blood glucose at 30 min (P<0.001 vs. all other treatments). The effect of DT-110 was also significantly different from alamandine (P<0.001). Metformin also had a significant effect on blood glucose which was significantly better than alamandine (P<0.001) overall. The overall effects of DT-110 and metformin were not found to be significantly different. These results indicate that while DT-110, an MRGD agonist that stimulates calcium mobilization, is a better glucose control agent than alamandine, an MRGD agonist that does not stimulate calcium mobilization. Metformin does not work through MRGD. The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features.

### Example 10

### Treatment of HUVEC with DT-110 Increases eNOS Expression

It has been shown that activation of MRGD by alamandine plays an important role in vasodilation by increasing nitric oxide (NO) production. NO produced by the endothelium is an important protective molecule in the vasculature and is mainly generated by the enzyme endothelial NO synthase (eNOS) in blood vessels. Enhancing the expression of eNOS has been considered as possible therapeutic strategy for reducing cardiovascular risk. To test the effect of DT-110 on eNOS expression, HUVEC were treated with vehicle, DT-110 or alamandine and eNOS mRNA levels determined at 30, 60 and 90 minutes. As shown in FIG. 15, at 30 minutes the levels of eNOS were the same for control, DT-110 and alamandine. Alamandine increased eNOS expression at 60 minutes but expression returned to control levels by 120 minutes. In contrast, DT-110 treatment showed increased eNOS expression at 60 and at 120 minutes. Because DT-109 increases eNOS expression it may also increase vasodilation in vivo as does alamandine, an endogenous hormone. Given that the increased eNOS expression caused by DT-110 is sustained longer that the increase caused by alamandine, DT-110 may be useful as a therapeutic agent for increasing blood levels of nitric oxide and for treating cardiovascular diseases or disorders, reducing cardiovascular risk, or treating cardiac symptoms, for example, hypertension, acute coronary syndrome, heart failure, or angina.

### Example 11

### DT-110 Stimulates Nitric Oxide Release in Primary Human Vascular Endothelium (HUVEC) Cells

To determine whether DT-110 stimulates Nitric Oxide (NO) release, real-time NO release was measured using the fluorescence indicator DAF-FM diacetate. In addition, dose response of NO with various concentrations of DT-110 was determined. DAF-FM diacetate is a reagent that is used to detect and quantify low concentrations of NO and is cell-permeant and passively diffuses across cellular membranes. Once inside cells, it is deacetylated by intracellular esterases to become DAF-FM. The fluorescence quantum yield of DAF-FM increases from about 0.005 to about 0.81 (about 160-fold) after reacting with NO. With excitation/emission maxima of 495/515 nm, DAF-FM can be detected by any instrument that can detect fluorescein, such as fluorescent microplate readers.

NO Release in HUVEC Cells

Briefly, HUVEC cells, previously plated in 96-well plate, were washed and the media changed to DMEM with 3% FBS for overnight incubation and cell starvation. On the day of the experiment, the cells were incubated with DAF-FM diacetate (Molecular Probes, catalog #D23842) at the final concentration of 7-10 nM for 30 minutes. Once loaded with dye, the cells were washed three times and left untreated vehicle control) or were treated with DT-110 at 4 mM for 10, 14, 19, 26, 32, 43, 57 and 68 minutes. Every treatment was performed in triplicate. The fluorescence was detected starting at 10 min with exc/em of 495 nm/515 nm.

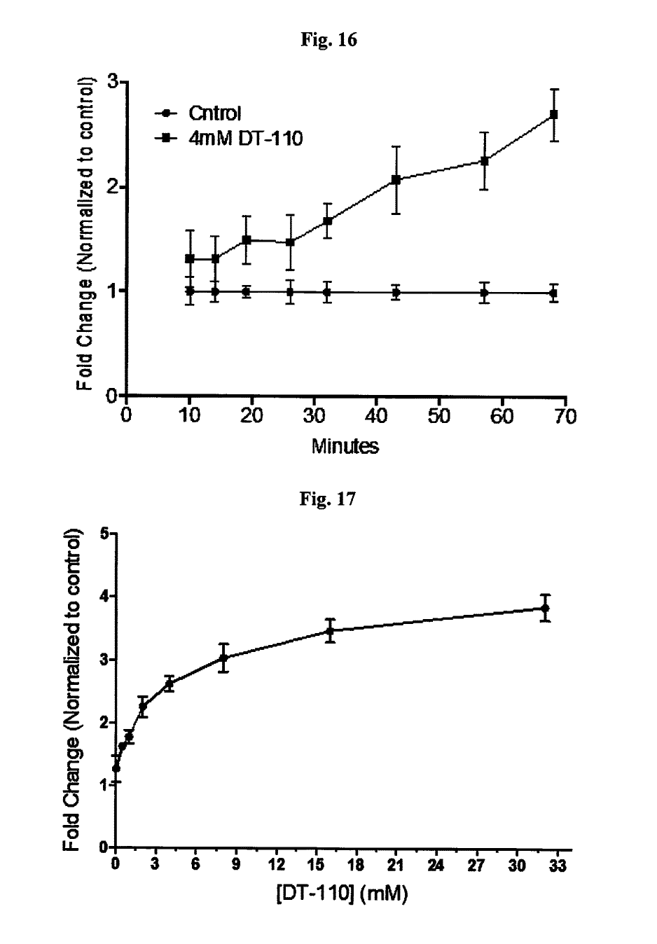

The background signal (fluorescence signal from cells that were not loaded with dye) was subtracted from the other measurements. The results from the vehicle control group was averaged and the results from all groups (including vehicle control) were then divided by that number to determine fold change. Results are shown in FIG. 16.

Dose Response

Briefly, HUVEC cells, previously plated in 96-well plate, were washed and the media changed to DMEM with 3% FBS for overnight incubation and cell starvation. On the day of the experiment, the cells were incubated with DAF-FM diacetate (Molecular Probes, catalog #D23842) at the final concentration of 7-10 nM for 30 minutes. Once loaded with dye, the cells were washed three times and left untreated vehicle control) or were treated with DT-110 at concentration of 0.05, 0.5, 1.0, 2.0, 4.0, 8.0, 16.0, and 32.0 mM for 60 minutes. Every treatment was performed in triplicate. The fluorescence was detected 60 minutes with exc/em of 495 nm/515 nm.

The background signal (fluorescence signal from cells that were not loaded with dye) was subtracted from the other measurements. The results from the vehicle control group were averaged and the results from all groups (including vehicle control) were then divided by that number to determine fold change. Results are shown in FIG. 17.

### Example 12

### Metabolic Analysis of MrgprD^{-/-} mice

Generation of MrgprD Knockout mice by RNA-Guided CRISPR/Cas9 Genome Editing

In order to determine the role of MrgprD in regulation of glucose homeostasis, MrgprD knockout mice (MrgprD^{-/-}) were generated by RNA-guided CRISPR/Cas9 genome editing. CRISPR/Cas9 system requires two in vitro synthesized RNAs to be delivered into embryos, one is a Cas9-encoding mRNA and other is a custom made single guide RNA (sgRNA or gRNA). sgRNA contains a targeting sequence (crRNA sequence) and a Cas9 nuclease-recruiting sequence (tracrRNA). The crRNA sequence is a 20-nucleotide sequence that is homologous to a region of interest in a gene used to direct Cas9 nuclease activity.

Cas9 mRNA was transcribed from plasmid pCas9 (JDS246, (Addgene, Cambridge Mass., plasmid #43861)) in vitro, caped and polyadenylated using the T7 mScript™ Standard mRNA Production System (Cellscript, Madison, Wis.). The sgRNA was designed using online Zifit software (zifit.partners.org/ZiFiT, ZiFiT Targeter Version 4.2). To construct the sgRNA expression vector, the crRNA sequence for MrgprD, GGGGATGGCAGGCAACTCAT (SEQ ID NO:1), was selected and the oligonucleotide was synthesized. The complement to this sequence, ATGAGTTGCCTGCCATCCCC (SEQ ID NO: 2) was also synthesized. The complementary pair of oligonucleotides were annealed and the double-stranded oligonucleotide was subcloned upstream of the tracrRNA in the gRNA cloning vector, pCRISPR. (DR274, (Addgene, Cambridge Mass., plasmid #42250)) Then the sgRNA was transcribed in vitro from the constructed vector by using T7-Scribe™ Standard RNA IVT Kit (Cellscript, Madison, Wis.). Cas9 mRNA and sgRNA were diluted in RNase-free TE buffer, and stored at −80° C. before microinjection into embryos.

Knockout mouse founders were generated by microinjection of Cas9 mRNA and sgRNA, described above, into C57BL/6J mouse embryos. For founder genotyping, skin samples were collected 4 weeks after birth, genomic DNA were extracted, and used as templates for PCR and sequencing. The founder mice with targeted sequence deletions were crossed with wildtype mice to generate heterozygous (MrgprD^{+/-}). DNAs were isolated from the tail samples and genotyping was performed. Further mating of heterozygous mice generated MrgprD^{-/-} (KO) mice.

The Fasting Glucose Levels in MrgprD^{-/-} mice are Significantly Higher Than that in Wildtype mice

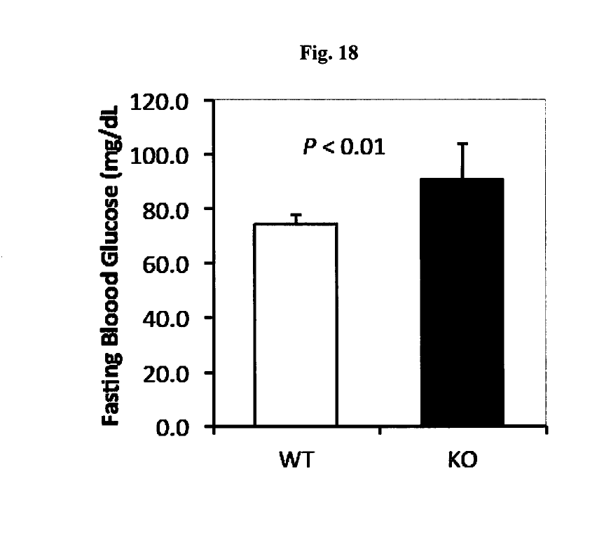

MrgprD^{-/-} (KO) mice, nine per group, were fasted for 16 hours and the fasting glucose levels were measured. The fasting blood glucose levels in KO mice were significantly higher than in wildtype mice suggesting that glucose homeostasis is impaired (P<0.01). The results are shown in FIG. 18. Data were analyzed by T-test and are expressed as the mean+standard deviation.

Effects of DT-109 on OGTT in MrgprD^{-/-} mice.

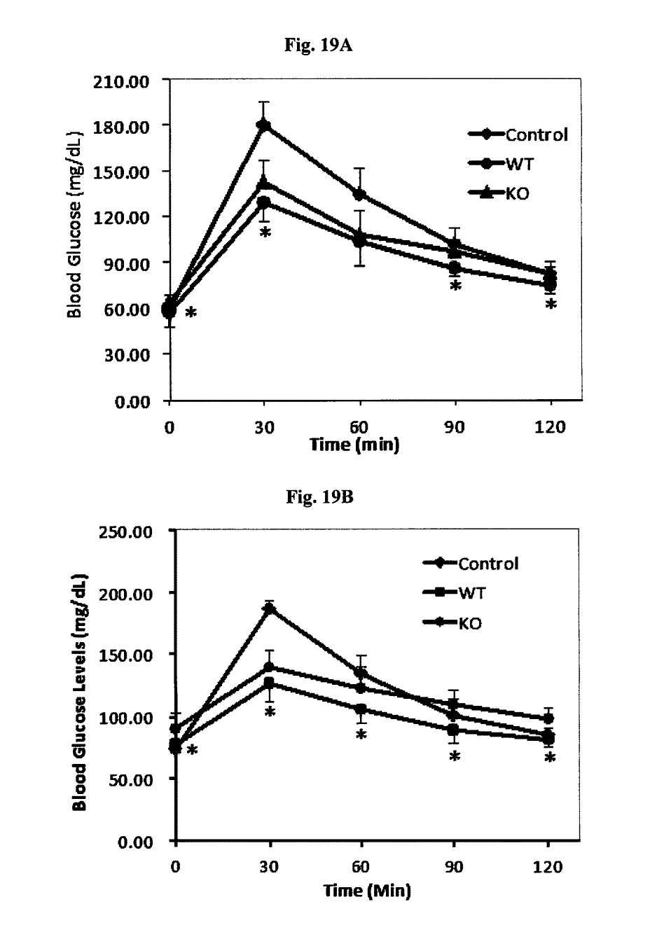

Oral Glucose tolerance tests (OGTT) were performed in C57BL/6J wildtype (WT) and MrgprD^{-/-} (KO) mice with or without DT-109 treatment. The mice were fasted overnight and their fasting glucose levels were measured. Then the mice were orally loaded with glucose (2 mg/g) in the control (n=10) and glucose (2 mg/g) plus DT-109 at 0.5 mg/g or 0.25 mg/g in the treated group (n=9). Blood glucose levels were measured at 30, 60, 90 and 120 minutes after glucose loading. As shown in FIGS. 19A and 19B, DT-109-induced reduction of blood glucose levels was significantly impaired in KO mice as compared with WT mice in both treatment groups. FIG. 19A shows the results with administration of 0.5 mg/g and FIG. 19B shows the results with administration of 0.25 mg/g DT-109 (*P<0.05, KO vs. WT).

Effects of DT-110 on OGTT in MrgprD^{-/-} mice.

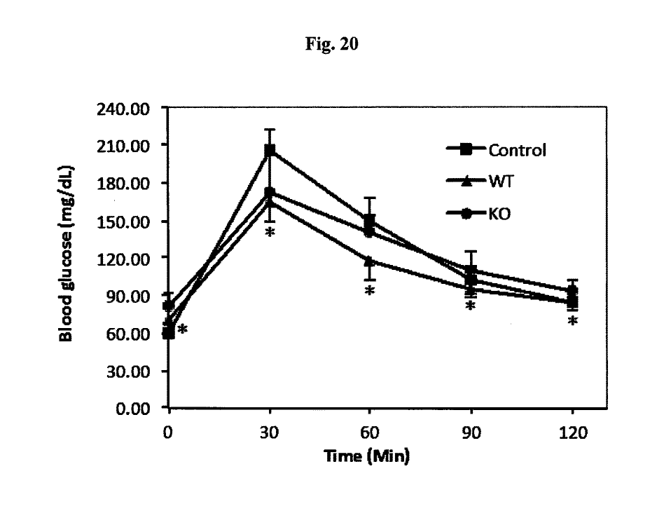

Oral Glucose tolerance tests (OGTT) were performed in C57BL/6J (WT) and MrgprD^{-/-} (KO) mice with or without DT-110 treatment. The mice were fasted overnight and their fasting glucose levels were measured. Then the mice were orally loaded with glucose (2 mg/g) in the control (n=10) and glucose (2 mg/g) plus 0.5 mg/g DT-110 in the treated group (n=9). Blood glucose levels were measured at 30, 60, 90 and 120 minutes after glucose loading. As shown in FIG. 20, DT-110-induced reduction of blood glucose levels in KO mice was significantly impaired as compared with WT mice (*P<0.05, KO vs. WT).

The description and specific examples, while indicating embodiments of the technology, are intended for purposes of illustration only and are not intended to limit the scope of the claims. Moreover, recitation of multiple embodiments having stated features is not intended to exclude other embodiments having additional features, or other embodiments incorporating different combinations of the stated features.

1. A method for identifying a candidate compound for the treatment of a condition associated with a dysregulation of blood glucose, the method comprising:
a) providing a cell expressing MRGD;
b) contacting the cell with a test compound;
c) determining whether the contacting causes a response of a GPCR-mediated signaling activity of MRGD in the cell contacted with the test compound; and
d) identifying the test compound as a candidate compound if the test compound causes a response of the GPCR-mediated signaling activity of MRGD.

2. The method according to claim 1, wherein the response is an increase in the concentration of intracellular cyclic adenosine monophosphate (cAMP), an increase in the concentration of intracellular inositol trisphosphate (IP3), or an increase in the concentration of intracellular calcium, or any combination thereof.

3. (canceled)

4. The method according to claim 1, wherein the method further comprises determining if the candidate compound stimulates GLP-1 secretion by an enteroendocrine L-cell.

5. The method according to claim 4, wherein the enteroendocrine L-cell is an STC-1 cell.

6. The method according to claim 1, the method further comprising administering the candidate compound to an MRGD wild-type mouse, and determining if the candidate compound lowers blood glucose levels.

7. The method according to claim 6, wherein determining if the candidate compound lowers blood glucose levels is evaluated using an oral glucose tolerance test wherein if the compound is lowers blood glucose levels the candidate compound is confirmed as a glucose lowering agent.

8. The method according to claim 7, the method further comprising administering the candidate compound to an MRGD knockout mouse, and determining if the candidate lowers blood glucose in the MRGD knockout mouse, wherein if the candidate compound does not significantly lower blood glucose levels the candidate compound is determined to act through the MRGD receptor.

9. The method according to claim 8, wherein determining if the candidate compound lowers blood glucose levels is evaluated using an oral glucose tolerance test.

10. The method according to claim 1, wherein the condition associated with the dysregulation of blood glucose is prediabetes or type-2 diabetes.

11. (canceled)

12. The method according to claim 1, wherein the condition associated with the dysregulation of blood glucose is a high blood glucose level, impaired glucose tolerance, or a high HbA1c level.

13. The method according to claim 1, wherein administration of the candidate compound to a subject reduces the level of blood glucose, reduces the level of HbA1c, or increases insulin sensitivity.

14. (canceled)

15. A method for treating a subject in need thereof by:
1) lowering blood glucose levels in a subject;
2) reducing HbA1c levels;
3) increasing GLP-1 levels;
4) increasing the secretion of insulin;
5) treating prediabetes; or
6) treating type-2 diabetes,
comprising administering to the subject a composition comprising an effective amount of an MRGD agonist, wherein the MRGD agonist is not β-alanine, GABA, GGL (DT-109), GLG, GLL, LLG, LGL, LGG, GGdL (DT-110), GdLG, GdLL, GLdL, GdLdL, dLLG, LdLG, dLdLG, dLGG, dLGL, LGdL, or dLGdL.

16-20. (canceled)

21. The method according to claim 15, wherein the MRGD agonist is Ang 1-7, alamandine or a combination thereof.

22. A method for treating a subject having a vascular or cardiovascular disease or disorder or reducing the risk or a vascular or cardiovascular disease or disorder, comprising administering to a subject in need thereof, a composition comprising a therapeutically effective amount of an MRGD agonist.

23. (canceled)

24. The method of claim 22, wherein administering the MRGD agonist:
1) increases the expression of eNOS;
2) increases the blood level nitric oxide; or
3) reduces weight,
in a subject in need thereof, comprising administering to the subject a composition comprising a therapeutically effective amount of an MRGD agonist.

25. (canceled)

26. (canceled)

27. The method according to claim 22, wherein the MRGD agonist is DT-109, DT-110, or a combination thereof.

28. (canceled)

29. The method according to claim 15, wherein the composition further comprises a pharmaceutically acceptable excipient.

30. The method according to claim 29, wherein the composition is administered by an oral, intravenous, intramuscular or subcutaneous route.

31. (canceled)

32. The method according to claim 22, wherein the composition further comprises a pharmaceutically acceptable excipient.

33. The method according to claim 32, wherein the composition is administered by an oral, intravenous, intramuscular or subcutaneous route.
